(19) United States
(12) Patent Application Publication
(10) Pub. No.: US 2004/0220424 A1
(43) Pub. Date: Nov. 4, 2004

(54) Process for preparing acids via alpha-chloroepoxy esters

(75) Inventors: Ann M. Diederich, Thomdale, PA (US); Vance J. Novack, Devon, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 10/859,384

(22) Filed: Jun. 2, 2004

Related U.S. Application Data
(63) Continuation of application No. 10/030,717, filed on Apr. 2, 2002, now abandoned.
Application No. 10/030,717 is a 371 of international application No. PCT/US00/21394, filed on Aug. 4, 2000.
(60) Provisional application No. 60/147,576, filed on Aug. 6, 1999.

(51) Int. Cl. C07C 231/02; C07C 69/76

(52) U.S. Cl. 560/81; 564/139

(57) ABSTRACT

This invention relates to a method for preparing certain acids of formula (I) via a chloroepoxy ester, which are useful as phosphodiesterase 4 inhibitors.

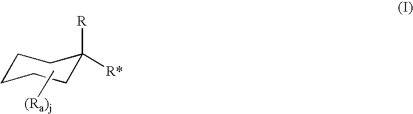

### AREA OF THE INVENTION

This invention relates to a method for preparing certain acids which are useful as phosphodiesterase 4 inhibitors. More specifically this invention relates to a method for converting cyclohexanones to cyclohexanoic acids via an alpha-haloepoxy ester.

### BACKGROUND OF THE INVENTION

The process of this invention relates to making compounds which are useful in treating diseases modulated by the isoforms of the phosphodiesterase 4 enzyme. The alpha-haloepoxy esters used in this process are unique compounds and useful in making acids which are known PDE 4 inhibitors which are useful, among other things, for treating pulmonary diseases such as chronic obstructive pulmonary disease (COPD) and asthma.

The compounds which are prepared by the methods of this invention and the intermediates disclosed herein are disclosed and described in the likes of U.S. Pat. No. 5,554,238 issued 3 Sep. 1996. That patent is incorporated herin by reference in full. Those compounds, particularly the 4-cyanocyclohexanoic acids, have marked effects on neutrophil activity, inhibiting neutrophil chemotaxis and degranulation in vitro. In animal models, those compounds reduce neutrophil extravasation from the circulation, pulmonary sequestration and the edematous responses to a number of inflammatory insults in vivo. They have been found to be useful in treating COPD in humans, and possibly in other mammalian species which suffer from COPD.

Herein there is provided a method for preparing certain of the phenyl-substituted cyclohexanoic acids, particularly those disclosed in U.S. Pat. No. 5,554,238 by starting with a cyclohexan-1-one and proceeding via a novel intermediate, an alpha-haloepoxy ester, to the acid analog of the ketone starting material.

### SUMMARY OF THE INVENTION

In a first aspect this invention relates to a process for preparing substituted cyclohexanoic acids of formula (I)

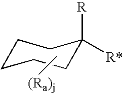

where Rₐ is a carbon-containing group optionally linked by oxygen, sulfur or nitrogen to the cyclohexyl ring and j is 1-10; and

R and R* are independently but not simultaneously hydrogen or C(O)E where E is OR₁₄ or SR₁₄;

which process comprises treating an epoxide of formula A with dimethyl sulfoxide and an alkali metal salt wherein formula A is:

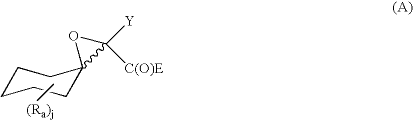

wherein E is OR₁₄ or SR₁₄ where R₁₄ is hydrogen or alkyl of 1-6 carbon atoms; Rₐ is the same as defined for Formula (I); and Y is Br, Cl, F or I.

More particularly this invention relates to a process for preparing compounds of formula IA

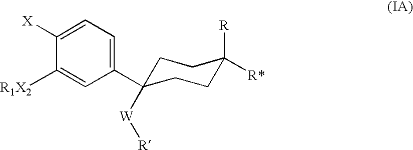

wherein:

R₁ is —(CR₄R₅)ₙC(O)O(CR₄R₅)ₘR₆, —(CR₄R₅)ₙC(O)NR₄(CR₄R₅)ₘR₆, —(CR₄R₅)ₙO(CR₄R₅)ₘR₆, or —(CR₄R₅)ᵣR₆ wherein the alkyl moieties are unsubstituted or substituted with one or more halogens;

m is 0 to 2;

n is 0 to 4;

r is 0 to 6;

R₄ and R₅ are independently selected hydrogen or C₁₋₂ alkyl;

R₆ is hydrogen, methyl, hydroxyl, aryl, halo substituted aryl, aryloxyC₁₋₃ alkyl, halo substituted aryloxyC₁₋₃ alkyl, indanyl, indenyl, C₇₋₁₁ polycycloalkyl, tetrahydrofuranyl, furanyl, tetrahydropyranyl, pyranyl, tetrahydrothienyl, thienyl, tetrahydrothiopyranyl, thiopyranyl, C₃₋₆ cycloalkyl, or a C₄₋₆ cycloalkyl containing one or two unsaturated bonds, wherein the cycloalkyl or heterocyclic moiety is unsubstituted or substituted by 1 to 3 methyl groups, one ethyl group, or an hydroxyl group;

provided that:

a) when R₆ is hydroxyl, then m is 2; or

b) when R₆ is hydroxyl, then r is 2 to 6; or

c) when R₆ is 2-tetrahydropyranyl, 2-tetrahydrothiopyranyl, 2-tetrahydrofuranyl, or 2-tetrahydrothienyl, then m is 1 or 2; or

d) when R₆ is 2-tetrahydropyranyl, 2-tetrahydrothiopyranyl, 2-tetrahydrofuranyl, or 2-tetrahydrothienyl, then r is 1 to 6;

e) when n is 1 and m is 0, then R₆ is other than H in —(CR₄R₅)ₙO(CR₄R₅)ₘR₆;

X is YR₂;

Y is O;

X₂ is O;

R₂ is —CH₃ or —CH₂CH₃, optionally substituted by 1 or more halogens;

R and R* are hydrogen or C(O)E wherein one of R or R* is always hydrogen and the other is always C(O)E where E is OR₁₄, or SR₁₄;

W is a bond or is alkenyl of 2 to 6 carbon atoms or alkynyl of 2 to 6 carbon atoms;

when W is a bond R′ is hydrogen, halogen, C₁₋₄ alkyl, CH₂NHC(O)C(O)NH₂, halo-substituted C₁₋₄ alkyl, CN, OR₈, CH₂OR₈, NR₈R₁₀, CH₂NR₈R₁₀, C(Z′)H, C(O)OR₈, or C(O)NR₈R₁₀; and

when W is alkenyl of 2 to 6 carbon atoms or alkynyl of 2 to 6 carbon atoms then R′ is COOR₁₄, C(O)NR₄R₁₄ or R₇;

R₇ is —(CR₄R₅)_{q}R₁₂ or C₁₋₆ alkyl wherein the R₁₂ or C₁₋₆ alkyl group is unsubstituted or substituted one or more times by: methyl or ethyl unsubstituted or substituted by 1-3 fluorines, or —F, —Br, —Cl, —NO₂, —NR₁₀R₁₁, —C(O)R₈, —CO₂R₈, —O(CH₂)₂₋₄OR₈, —O(CH₂)_{q}R₈, —CN, —C(O)NR₁₀R₁₁, —O(CH₂)_{q}C(O)NR₁₀R₁₁, —O(CH₂)_{q}C(O)R₉, —NR₁₀C(O)NR₁₀R₁₁, —NR₁₀C(O)R₁₁, —NR₁₀C(O)OR₉, —NR₁₀C(O)R₁₃, —C(NR₁₀)NR₁₀R₁₁, —C(NCN)NR₁₀R₁₁, —C(NCN)SR₉, —NR₁₀C(NCN)SR₉, —NR₁₀C(NCN)NR₁₀R₁₁, —NR₁₀S(O)₂R₉, —S(O)_{m′}R₉, —NR₁₀C(O)C(O)NR₁₀R₁₁, —NR₁₀C(O)C(O)R₁₀, or R₁₃;

q is 0, 1, or 2;

R₁₂ is R₁₃, C₃-C₇ cycloalkyl, or an unsubstituted or substituted aryl or heteroaryl group selected from the group consisting of (2-, 3- or 4-pyridyl), pyrimidyl, pyrazolyl, (1- or 2-imidazolyl), pyrrolyl, piperazinyl, piperidinyl, morpholinyl, furanyl, (2- or 3-thienyl), quinolinyl, naphthyl, and phenyl;

R₈ is independently selected from hydrogen or R₉;

R₉ is C₁₋₄ alkyl optionally substituted by one to three fluorines;

R₁₀ is OR₈ or R₁₁;

R₁₁ is hydrogen, or C₁₋₄ alkyl unsubstituted or substituted by one to three fluorines; or when R₁₀ and R₁₁ are as NR₁₀R₁₁ they may together with the nitrogen form a 5 to 7 membered ring comprised of carbon or carbon and one or more additional heteroatoms selected from O, N, or S;

R₁₃ is a substituted or unsubstituted heteroaryl group selected from the group consisting of oxazolidinyl, oxazolyl, thiazolyl, pyrazolyl, triazolyl, tetrazolyl, imidazolyl, imidazolidinyl, thiazolidinyl, isoxazolyl, oxadiazolyl, and thiadiazolyl, and where R₁₃ is substituted on R₁₂ or R₁₃ the rings are connected through a carbon atom and each second R₁₃ ring may be unsubstituted or substituted by one or two C₁₋₂ alkyl groups unsubstituted or substituted on the methyl with 1 to 3 fluoro atoms; and

R₁₄ is hydrogen or C₁₋₆ alkyl;

which process comprises treating an epoxide of formula A with dimethyl sulfoxide and an alkali metal salt wherein formula A is:

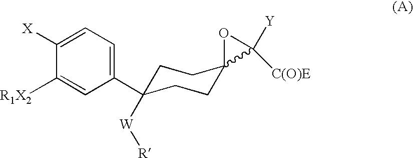

wherein X; R₁X₂; W; E; R′; and R₁₄ are the same as defined for Formula (IA); and Y is Br, Cl, F or I.

In a second aspect this invention relates to a process for making the epoxide of Formula (A)

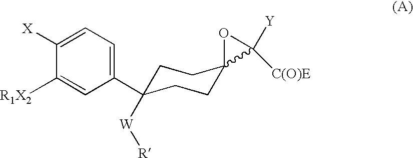

where R₁X₂, X, W, R′ and Y are the same as for Formula (IA) and the R₁₄ in E is C₁₋₆alkyl; which process comprises treating a ketone of Formula (B) with an alkyldihaloacetate or alkyldihalothioacetate in a polar aprotic solvent and optionally saponifying the resulting epoxy ester or thioester,

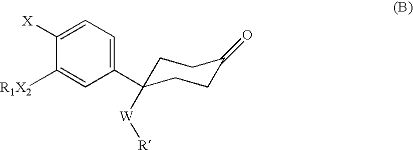

wherein X, R₁X₂, W and R′ are the same as in Formula (A).

In a further aspect, this invention relates to a method for enriching the cis form of Formula (I) or (IA) where one of R or R* is C(O)OH or C(O)SH and the other is hydrogen in a mixture of cis and trans isomers. The method comprises esterifying the acid or thioacid or converting them to a mixed anhydride, if they are not already in that form, then treating the ester, etc, with an alkoxide base for a time sufficient to give a ratio of cis to trans isomers which is at least 4:1, perferably 7:1 or greater.

In yet another aspect, this invention relates to the haloepoxy acids and haloepoxy esters, thioesters and mixed anhydrides of formula A.

### DETAILED DESCRIPTION OF THE INVENTION

This invention provides a means for preparing cyclohexanoic acids. In particular it relates to a method for preparing cyclohexanoic acids which are phosphodiesterase 4 inhibitors as more fully disclosed in U.S. Pat. No. 5,554,238. The invention can also be used to prepare any cyclohexanoic acid and for enriching the cis form of a cyclohexanoic acid in a mixture of cis and trans isomers.

As regards the preferred substituents on Formulas (IA), (A) and (B), for R₁ they are CH₂-cyclopropyl or C₄₋₆ cycloalkyl. Preferred R₂ groups are a C₁₋₂ alkyl unsubstituted or substituted by 1 or more halogens. The halogen atoms are preferably fluorine and chlorine, more preferably fluorine. More preferred R₂ groups are those wherein R₂ is methyl, or a fluoro-substituted alkyl group, specifically a C₁₋₂ alkyl such as a —CF₃, —CHF₂, or —CH₂CHF₂. Most preferred are the —CHF₂ and —CH₃ moieties. Most preferred are those compounds wherein R₁ is —CH₂-cyclopropyl, cyclopentyl, 3-hydroxycyclopentyl, methyl or CHF₂ and R₂ is CF₂H or CH₃. Preferably the R₁₄ group will be methyl, ethyl or hydrogen. In formula (IA), methyl is the most preferred R₁₄ group, and in Formula (I), it is methyl or hydrogen. Particularly preferred are those compounds where R₁ is cyclopentyl and R₂ is CH₃.

As regards W, the preferred embodiment is where W is a bond, ethylenyl, or —C≡C—. When W is a bond, the preferred R′ group is CN. And when W is ethylenyl, —C≡C— the preferred R′ group is hydrogen.

The most preferred compound of Formula (IA) made by the process of this invention is cis-[4-cyano-4-(3-cyclopentyloxy-4-methoxyphenyl)cyclohexane-1-carboxylic acid].

In regards to the epoxides, the lower alkyl chloroepoxy esters, thioesters and their corresponding acids are preferred. Methyl and ethyl are the most preferred ester-forming groups. The most preferred epoxides are methyl 2-chloro-6-cyano-6-[3-(cyclopentyloxy)-4-methoxyphenyl]-1-oxaspiro[2.5]octane-2-carboxylate and 2-chloro-6-cyano-6-[3-(cyclopentyloxy)-4-methoxyphenyl]-1-oxaspiro[2.5]octane-2-carboxylic acid.

Scheme I illustrates the conversion of a ketone of Formula (B) to the ester or acid of Formula (IA).

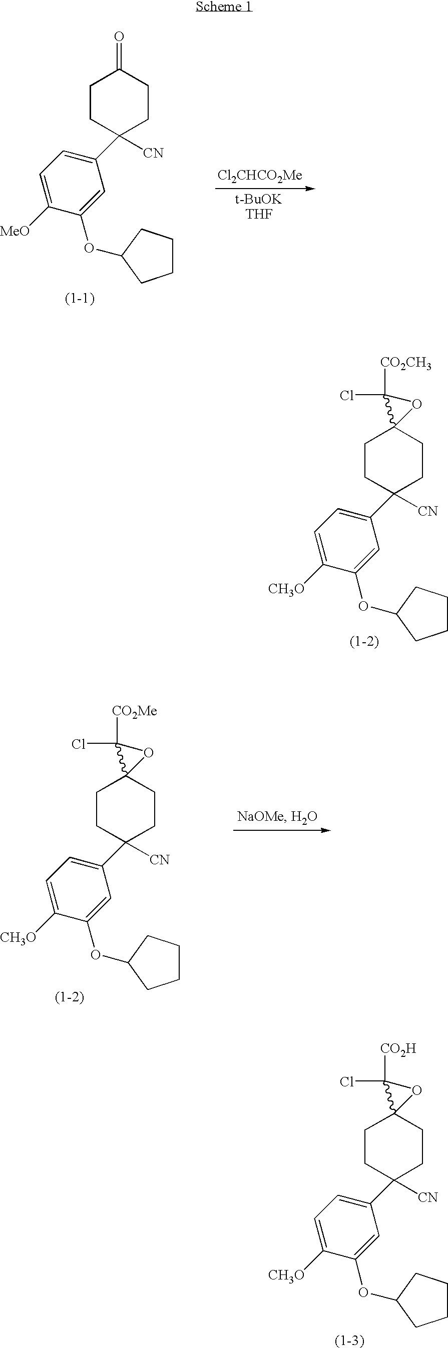

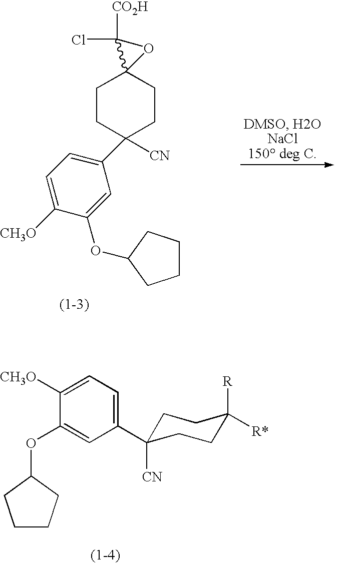

In compound 1-4, R and R* are hydrogen or C(O)OH, but R and R* are not both hydrogen or C(O)OH simultaneously.

The ketone starting material (1-1) can be prepared by the methods set out in U.S. Pat. No. 5,554,238 or U.S. Pat. No. 5,449,686. Forming the epoxide (1-2) is accomplished by treating the ketone with 1.1 to 2 equivalents of a lower alkyldihaloacetate using a polar non-protic solvent. “Lower alkyl” here means an alkyl radical having 1-6 carbon atoms. It is preferred to use about 1.5 equivalents of the acetate, and teterahydrofuran as the solvent. First the ketone (1-1) and the actetate are dissolved in the solvent. This solution is cooled to between −10 and +10° C. and an organic base is added in a molar excess (e.g. 1.1 to 2 equivalents, preferably about 1.5 equivalents). Herein an alkali metal t-butoxide is the preferred base, particularly potassium tert-butoxide. The temperature is kept at within the −10 to +10° C. range during the addition of the base and for some short period, 10 minutes to 45 minute thereafter. Product (1-2) is recovered by conventional means.

The ester (1-2) is then saponified using a base. This can be accomplished by any number of bases using conventional techniques. Herein this reaction is effected by treating the α-chloroepoxyester with sodium methoxide using a low molecular-weight alcohol and water as the solvent. A substantial molar excess of the base and solvent is used. For example a 5-fold excess of the base can be used and about a 10-fold excess of water. The ester is charged to a reaction vessel, dissolved in the alcohol, base is added and then the water is added The reaction goes to completion rapidly at room temperature, about 5 to 30 minutes. Product, the acid, is recovered by conventional means. Since it, the α-chloroepoxy acid (1-3), is relatively unstable it is preferred to immediately treat the epoxide with a reactant which opens the ring to give the acid.

Herein the epoxy acid (1-3) is rearranged to give 1-4 using dimethyl sulfoxide and an alkali metal salt. Water is used as a co-solvent. The alkali metal salt may be LiCl, KCl or NaCl, or the corresponding flouride and bromide salts LiF. KF, NaF, LiBr, KBr, and NaBr. By way of more specific example, the chloroepoxy acid is dissolved in dimethyl sulfoxide and water and a small amount of a sodium chloride is added to the reaction pot which is then heated for several hours. A preferred set of reactants and conditions is one where about a 10-fold excess of DMSO (by weight/volume) is used to dissolve the acid and a small amount of water and a salt such as sodium chloride is added. This solution is heated to between about 125 and 175° C. for 2-5 hours; preferably the solution is heated to about 150° C. for 3.5 hours or so. This reaction gives the cyclohexanoic acid as a mixture of the cis and trans isomers in about a 1-1 ratio.

Enrichment of the cis isomer in the mixture of cis and trans isomers obtained from the just-described reaction is accomplished by activating it by, for example, forming an ester or mixed anhydride, and then treating the ester with an alkoxide base. This technique can be applied with satisfactory results to any preparation where one has a mixture of isomers and wishes to enrich the cis form of the isomer in that mixture. By way of example, the technique used here is to esterify the acid using an acid and a lower alkanol to form the ester of the alkanol. Methanol is most preferred. This mixture is then treated with t-butanol and its alkali metal salt for an extended period, for between 5 and 24 hours for example; a preferred time is about 12 hours. This latter step results in an enrichment of the cis form of the product; the equilibration process gives the preferred cis form of the acid.

An alternative process is to combine the step of opening of the epoxy acid, really a decarboxylation, with the esterification step by using a lower alkanol or lower thioalkanol (1-6 carbons) as the co-solvent instead of water. The re-equilibration can be effected by adding the appropriate alcohol and its alkali metal salt to the reaction flask once the ester has been formed from the α-haloepoxy acid without isolating the ester. For example methanol rather than water can be used as the solvent for the dimethyl sulfoxide/salt reaction. If this is done, one obtains the methyl ester as the product, rather than the acid obtained when water is used as the solvent. However if methanol or another low boiling-point alkanol is used, a pressurizable reaction vessel must be employed since the solution must be heated to about 150° C. to effect the decarboxylation, at which temperature the methanol would be mostly vaporized if the reaction was run at 1 atmosphere of pressure. A preferred approach is to run the reaction using methanol in a pressurized container, cooling the reaction mixture to about room temperature, and adding the likes of t-butanol and its alkali metal salt to effect the coversion of the trans form to the cis isomer.

By way of further illustration, but without intending to be limited in any way, the following illustrative examples are provided.

### EXAMPLES

### Example 1

### Preparation of methyl 2-chloro-6-cyano-6-[3-(cyclopentyloxy)-4-methoxyphenyl]-1-oxaspiro[2.5]octane-2-carboxylate

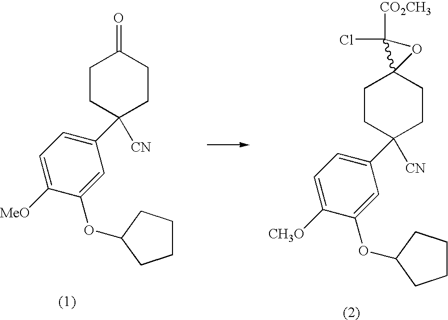

A 100 mL round-bottom flask was charged with 4-cyano-4-(3-cyclopentyloxy-4-methoxyphenyl)cyclohexan-1-one (1)(4.0 g, 12.8 mmole, 1.0 eq), methyldichloroacetate (2.74 g, 1.98 mL, 19.1 mmole, 1.5 eq), and tetrahydrofuran (THF, 40 mL). The solution was cooled to 0° C. in an ice bath, then potassium tert-butoxide was added (19.1 mL, 19.1 mmole of a 1M solution in THF) while maintaining the temperature below 5° C. (about 25 minutes). The reaction was deemed complete at the end by TLC (hexanes/ethyl acetate @3/1, silica gel plates), then was poured into ethyl acetate and 5% HCl for an extractive workup. The layers were separated and the water layer was extracted with ethyl acetate twice. The combined ethyl acetate layers were extracted with 5% sodium bicarbonate and with brine. The ethyl acetate layer was concentrated under vacuum to a yellow oil. The oil was dissolved in 3/1 hexanes/ethyl acetate and filtered through 1.5″ of flash silica gel. Concentration produced the product methyl 2-chloro-6-cyano-6-[3-(cyclopentyloxy)-4-methoxyphenyl]-1-oxaspiro[2.5]octane-2-carboxylate as a clear, colorless oil. The molecular weight and structure of the product was confirmed to be the methyl α-chloroepoxy ester by mass spec.

### Example 2

### Preparation of 2-chloro-6-cyano-6-[3-(cyclopentyloxy)-4-methoxyphenyl]-1-oxaspiro[2.5]octane-2-carboxylic acid

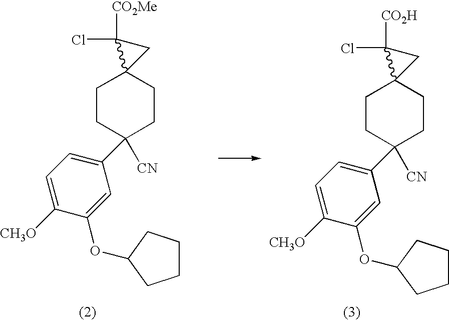

A 50 mL flask was charged with the chloroepoxyester (2) (3.0 g, 4.77 mmole), 30 mL of methanol, sodium methoxide (5.16 g of 25 wt % solution in methanol, 23.9 mmole) and water (0.8 g, 44 mmole). The solution was stirred for 10 minutes and the reaction was deemed complete by TLC (hexanes/ethyl acetate @3/1, silica gel plates). The reaction was poured into an addition funnel containing 100 mL of 1% HCl and 100 mL of t-butylmethyl ether. The organic layer was extracted once with water and once with brine, then was concentrated to an oil under reduced pressure. The product 2-chloro-6-cyano-6-[3-(cyclopentyloxy)-4-methoxyphenyl]-1-oxaspiro[2.5]octane-2-carboxylic acid was confirmed by mass spectral analysis.

### Example 3

### Preparation of cis-[4-cyano-4-(3-cyclopentyloxy-4-methoxyphenyl)cyclohexane-1-carboxylic acid]

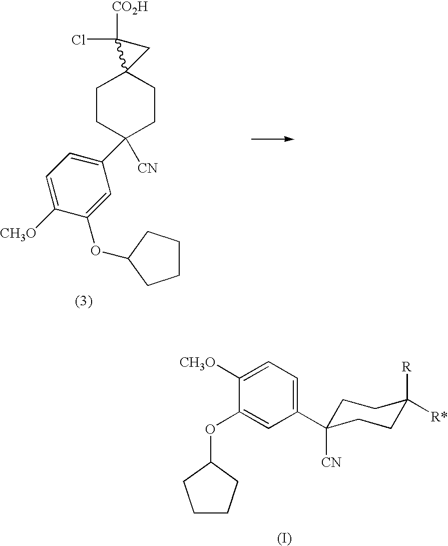

In this example, either R or R* can be C(O)OH; the other group must be hydrogen.

Freshly prepared chloroepoxyacid (3) (2.79 mmole) was treated with dimethyl sulfoxide (7.5 mL), water (0.5 mL), and NaCl (50 mg). The solution was heated to 150° C. for 3.5 hours. The reaction was followed by HPLC (15 cm Supelcocil, ACN/water/TFA [40/60/0.1] 1.5 ml/min, 215 nm UV, trans form—at 10.6 min and cis form at 11.3 min). The yield was calculated using weighted assay. The yield was 59% for the two isomers in a ratio of one-to-one.

### Example 4

### Enrichment of the Cis Isomer in a Cis/Trans Mixture of [4-cyano-4-(3-cyclopentyloxy-4-methoxyphenyl)cyclohexane-1-carboxylic acid]

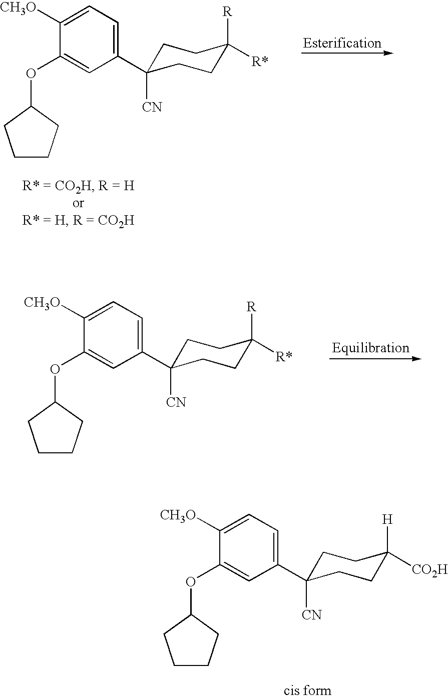

The isomeric mixture obtained in the previous step was dissolved in 10 mL of methanol. p-Toluenesulfonic acid (0.1 g) was added and the reaction was refluxed for 12 hours to form the methyl esters. The reaction was diluted with ethyl acetate and water. The layers were separated, then the organic layer was concentrated. The oil was dissolved in about 10 mL of tBuOH and then 7.5 mL of potassium t-butoxide (1M in t-BuOH) was added for the equilibration. After stirring overnight, a small sample was treated with water and the ratio of cis to trans isomers of [4-cyano-4-(3-cyclopentyloxy-4-methoxyphenyl)cyclohexane-1-carboxylic acid] was calculated as 9.6/1 by HPLC (15 cm Supelcocil, ACN/water/TFA [40/60/0.1] 1.5 mL/min, 215 nm UV, trans form at 10.6 min and cis form at 11.3 min). The reaction was quenched by adding 1% HCl and ethyl acetate to extract. The layers were separated and the organic layer was extracted once with water. The product layer was concentrated and then treated with ethyl acetate. The product was precipitated by adding about one volume of hexanes. No trans form was detected in the product.

This reaction was also run using NaH under the same conditions. It gave an 8:1 ratio of cis:trans isomers. When the same reaction was run using NaH in ethanol (methyl ester) a 7:1 ratio was obtained. Using the ethyl ester rather than the methyl ester as the substrate, and NaH and ethanol, a 10:1 ratio was obtained.

### Example 5

### One-Pot Preparation of the Cis-[4-cyano-4-(3-cyclopentyloxy-4-methoxyphenyl)cyclohexane-1-carboxylic acid] from Methyl 2-chloro-6-cyano-6-[3-(cyclopentyloxy)-4-methoxyphenyl]-1-oxaspiro[2.5]octane-2-carboxylate

Chloroepoxyester (0.72 g purified, 1.71 mmole) in methanol (5 mL) was treated with sodium methoxide (1.42 g of 25% wt solution in methanol, 6.5 mmole) and water (0.5 mL) and stirred for 15 minutes. The reaction was quenched with t-butylmethyl ether and 1% HCl. The bottom layer was removed, then the organic layer was washed three times with water. The organic layer was concentrated under reduced pressure, then the water was azeotroped by adding methanol and reconcentrating.

Dimethylsulfoxide (7 mL), sodium chloride (0.5 g) and methanol (5 mL) were added. The contents were then heated under pressure to 150° C. for 1.5 hours. The HPLC (15 cm Supelcocil, ACN/water/TFA [40/60/0.1] 1.5 mL/min, 215 nm UV) showed the isomeric mixture of esters and acids at 10.5/1 (esters/acids). The reaction was cooled, then 10 mL of t-BuOH and 0.20 g of t-BuOK was added The solution was stirred overnight to give a 7/1 ratio of cis/trans isomers. The reaction was worked up with 1% HCl and t-butylmethyl ether. The layers were separated and the organic layer was concentrated to an oil. The oil was dissolved in a minimum amount of warm ethyl acetate, and the product was precipitated by adding hexanes, cooled to 0° C., then filtered. The product was a light tan solid; no trans isomer was detected.

1. A process for preparing substituted cyclohexanoic acids of formula (I) 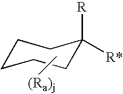
where Rₐ is a carbon-containing group optionally linked by oxygen, sulfur or nitrogen to the cyclohexyl ring and j is 1-10; and
R and R* are independently but not simultaneously hydrogen or C(O)E where E is OR₁₄ or SR₁₄ where R₁₄ is hydrogen or alkyl of 1-6 carbon atoms;
which process comprises treating an epoxide of formula A with dimethyl sulfoxide and an alkali metal salt, wherein formula A is: 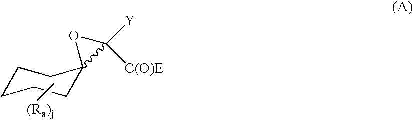
wherein E is OR₁₄ or SR₁₄ where R₁₄ is hydrogen or alkyl of 1-6 carbon atoms; Rₐ is the same as defined for Formula (I); and Y is Br, Cl, F or I.

2. A process for preparing compounds of formula IA 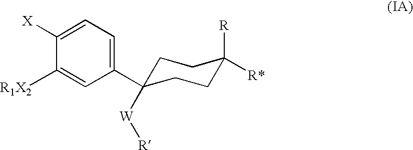
wherein:
R₁ is —(CR₄R₅)ₙC(O)O(CR₄R₅)ₘR₆, —(CR₄R₅)ₙC(O)NR₄(CR₄R₅)ₘR₆, —(CR₄R₅)ₙO(CR₄R₅)ₘR₆, or —(CR₄R₅)ᵣR₆ wherein the alkyl moieties are unsubstituted or substituted with one or more halogens;
m is 0 to 2;
n is 0 to 4;
r is 0 to 6;
R₄ and R₅ are independently selected hydrogen or C₁₋₂ alkyl;
R₆ is hydrogen, methyl, hydroxyl, aryl, halo substituted aryl, aryloxyC₁₋₃ alkyl, halo substituted aryloxyC₁₋₃ alkyl, indanyl, indenyl, C₇₋₁₁ polycycloalkyl, tetrahydrofuranyl, furanyl, tetrahydropyranyl, pyranyl, tetrahydrothienyl, thienyl, tetrahydrothiopyranyl, thiopyranyl, C₃₋₆ cycloalkyl, or a C₄₋₆ cycloalkyl containing one or two unsaturated bonds, wherein the cycloalkyl or heterocyclic moiety is unsubstituted or substituted by 1 to 3 methyl groups, one ethyl group, or an hydroxyl group;
provided that:
a) when R₆ is hydroxyl, then m is 2; or
b) when R₆ is hydroxyl, then r is 2 to 6; or
c) when R₆ is 2-tetrahydropyranyl, 2-tetrahydrothiopyranyl, 2-tetrahydrofuranyl, or 2-tetrahydrothienyl, then m is 1 or 2; or
d) when R₆ is 2-tetrahydropyranyl, 2-tetrahydrothiopyranyl, 2-tetrahydrofuranyl, or 2-tetrahydrothienyl, then r is 1 to 6;
e) when n is 1 and m is 0, then R₆ is other than H in —(CR₄R₅)ₙO(CR₄R₅)ₘR₆;
X is YR₂;
Y is O;
X₂ is O;
R₂ is —CH₃ or —CH₂CH₃, optionally substituted by 1 or more halogens;
R and R* are hydrogen or C(O)E wherein one of R or R* is always hydrogen and the other is always C(O)E where E is OR₁₄, or SR₁₄;
W is a bond or is alkenyl of 2 to 6 carbon atoms or alkynyl of 2 to 6 carbon atoms;
when W is a bond R′ is hydrogen, halogen, C₁₋₄ alkyl, CH₂NHC(O)C(O)NH₂, halo-substituted C₁₋₄ alkyl, CN, OR₈, CH₂OR₈, NR₈R₁₀, CH₂NR₈R₁₀, C(Z′)H, C(O)OR₈, or C(O)NR₈R₁₀; and
when W is alkenyl of 2 to 6 carbon atoms or alkynyl of 2 to 6 carbon atoms then R′ is COOR₁₄, C(O)NR₄R₁₄ or R₇;
R₇ is —(CR₄R₅)_{q}R₁₂ or C₁₋₆ alkyl wherein the R₁₂ or C₁₋₆ alkyl group is unsubstituted or substituted one or more times by: methyl or ethyl unsubstituted or substituted by 1-3 fluorines, —F, —Br, —Cl, —NO₂, —NR₁₀R₁₁, —C(O)R₈, —CO₂R₈, —O(CH₂)₂₋₄OR₈, —O(CH₂)_{q}R₈, —CN, —C(O)NR₁₀R₁₁, —O(CH₂)_{q}C(O)NR₁₀R₁₁, —O(CH₂)_{q}C(O)R₉, —NR₁₀C(O)NR₁₀R₁₁, —NR₁₀C(O)R₁₁, —NR₁₀C(O)OR₉, —NR₁₀C(O)R₁₃, —C(NR₁₀)NR₁₀R₁₁, —C(NCN)NR₁₀R₁₁, —C(NCN)SR₉, —NR₁₀C(NCN)SR₉, —NR₁₀C(NCN)NR₁₀R₁₁, —NR₁₀S(O)₂R₉, —S(O)_{m′}R₉, —NR₁₀C(O)C(O)NR₁₀R₁₁, —NR₁₀C(O)C(O)R₁₀, or R₁₃;
q is 0, 1, or 2;
R₁₂ is R₁₃, C₃-C₇ cycloalkyl, or an unsubstituted or substituted aryl or heteroaryl group selected from the group consisting of (2-, 3- or 4-pyridyl), pyrimidyl, pyrazolyl, (1- or 2-imidazolyl), pyrrolyl, piperazinyl, piperidinyl, morpholinyl, furanyl, (2- or 3-thienyl), quinolinyl, naphthyl, and phenyl;
R₈ is independently selected from hydrogen or R₉;
R₉ is C₁₋₄ alkyl optionally substituted by one to three fluorines;
R₁₀ is OR₈ or R₁₁;
R₁₁ is hydrogen, or C₁₋₄ alkyl unsubstituted or substituted by one to three fluorines; or when R₁₀ and R₁₁ are as NR₁₀R₁₁ they may together with the nitrogen form a 5 to 7 membered ring comprised of carbon or carbon and one or more additional heteroatoms selected from O, N, or S;
R₁₃ is a substituted or unsubstituted heteroaryl group selected from the group consisting of oxazolidinyl, oxazolyl, thiazolyl, pyrazolyl, triazolyl, tetrazolyl, imidazolyl, imidazolidinyl, thiazolidinyl, isoxazolyl, oxadiazolyl, and thiadiazolyl, and where R₁₃ is substituted on R₁₂ or R₁₃ the rings are connected through a carbon atom and each second R₁₃ ring may be unsubstituted or substituted by one or two C₁₋₂ alkyl groups unsubstituted or substituted on the methyl with 1 to 3 fluoro atoms; and
R₁₄ is hydrogen or C₁₋₆ alkyl;
which process comprises treating an epoxide of Formula A 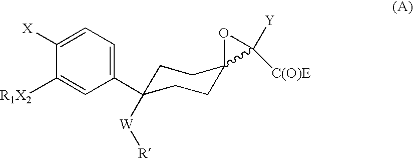
with dimethyl sulfoxide and an alkali metal salt;
wherein X; R₁X₂; W; E; R′; R₁₄ are the same as defined for Formula (IA); and Y is Br, Cl, F or I.

3. The process of claim 2 wherein alkali metal salt is LiCl, KCl, or NaCl and the reaction is carried out at between about 125-175° C. or 2-5 hours.

4. The process of claim 3 wherein the compound of formula A is one where R₁ is CH₂-cyclopropyl, CH₂-C₅₋₆ cycloalkyl, or C₄₋₆ cycloalkyl and R₂ is C₁₋₂ alkyl unsubstituted or substituted by 1 or more halogens.

5. The process of claim 4 wherein, in the compound of formula A, W is a bond or —C≡C— and R′ is CN or 2-aminopyrimidin-5-yl, R₁ is cyclopentyl and R₂ is CH₃.

6. The process of claim 5 wherein, in the compound of formula A, W is a bond, R′ is CN.

7. The process of claim 2 wherein at 10-fold excess of dimethyl sulfoxide is used, the salt is sodium chloride and the reaction is heated to about 150° C. for about 3.5 hours.

8. A process for preparing an epoxide of Formula (A) 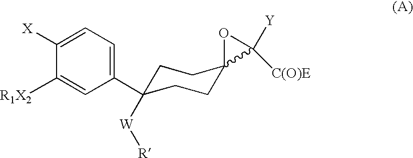
wherein:
R₁ is —(CR₄R₅)ₙC(O)O(CR₄R₅)ₘR₆, —(CR₄R₅)ₙC(O)NR₄(CR₄R₅)ₘR₆, —(CR₄R₅)ₙO(CR₄R₅)ₘR₆, or —(CR₄R₅)ᵣR₆ wherein the alkyl moieties unsubstituted or substituted with one or more halogens;
m is 0 to 2;
n is 0 to 4;
r is 0 to 6;
R₄ and R₅ are independently selected hydrogen or C₁₋₂ alkyl;
R₆ is hydrogen, methyl, hydroxyl, aryl, halo substituted aryl, aryloxyC₁₋₃ alkyl, halo substituted aryloxyC₁₋₃ alkyl, indanyl, indenyl, C₇₋₁₁ polycycloalkyl, tetrahydrofuranyl, furanyl, tetrahydropyranyl, pyranyl, tetrahydrothienyl, thienyl, tetrahydrothiopyranyl, thiopyranyl, C₃₋₆ cycloalkyl, or a C₄₋₆ cycloalkyl containing one or two unsaturated bonds, wherein the cycloalkyl or heterocyclic moiety is unsubstituted or substituted by 1 to 3 methyl groups, one ethyl group, or an hydroxyl group;
provided that:
a) when R₆ is hydroxyl, then m is 2; or
b) when R₆ is hydroxyl, then r is 2 to 6; or
c) when R₆ is 2-tetrahydropyranyl, 2-tetrahydrothiopyranyl, 2-tetrahydrofuranyl, or 2-tetrahydrothienyl, then m is 1 or 2; or
d) when R₆ is 2-tetrahydropyranyl, 2-tetrahydrothiopyranyl, 2-tetrahydrofuranyl, or 2-tetrahydrothienyl, then r is 1 to 6;
e) when n is 1 and m is 0, then R₆ is other than H in —(CR₄R₅)ₙO(CR₄R₅)ₘR₆;
X is YR₂;
Y is O;
X₂ is O;
W is a bond or is alkenyl of 2 to 6 carbon atoms or alkynyl of 2 to 6 carbon atoms;
when W is a bond, R′ is hydrogen, halogen, C₁₋₄ alkyl, CH₂NHC(O)C(O)NH₂, halo-substituted C₁₋₄ alkyl, CN, OR₈, CH₂OR₈, NR₈R₁₀, CH₂NR₈R₁₀, C(Z′)H, C(O)OR₈, or C(O)NR₈R₁₀; and
when W is alkenyl of 2 to 6 carbon atoms or alkynyl of 2 to 6 carbon atoms, R′ is R′ is COOR₁₄, C(O)NR₄R₁₄ or R₇;
R₂ is —CH₃ or —CH₂CH₃, optionally substituted by 1 or more halogens; and
R₇ is —(CR₄R₅)_{q}R₁₂ or C₁₋₆ alkyl wherein the R₁₂ or C₁₋₆ alkyl group is unsubstituted or substituted one or more times by: methyl or ethyl unsubstituted or substituted by 1-3 fluorines, —F, —Br, —Cl, —NO₂, —NR₁₀R₁₁, —C(O)R₈, —CO₂R₈, —O(CH₂)₂₋₄OR₈, —O(CH₂)_{q}R₈, —CN, —C(O)NR₁₀R₁₁, —O(CH₂)_{q}C(O)NR₁₀R₁₁, —O(CH₂)_{q}C(O)R₉, —NR₁₀C(O)NR₁₀R₁₁, —NR₁₀C(O)R₁₁, —NR₁₀C(O)OR₉, —NR₁₀C(O)R₁₃, —C(NR₁₀)NR₁₀R₁₁, —C(NCN)NR₁₀R₁₁, —C(NCN)SR₉, —NR₁₀C(NCN)SR₉, —NR₁₀C(NCN)NR₁₀R₁₁, —NR₁₀S(O)₂R₉, —S(O)_{m′}R₉, —NR₁₀C(O)C(O)NR₁₀R₁₁, —NR₁₀C(O)C(O)R₁₀, or R₁₃;
q is 0, 1, or 2;
R₁₂ is R₁₃, C₃-C₇ cycloalkyl, or an unsubstituted or substituted aryl or heteroaryl group selected from the group consisting of (2-, 3- or 4-pyridyl), pyrimidyl, pyrazolyl, (1- or 2-imidazolyl), pyrrolyl, piperazinyl, piperidinyl, morpholinyl, furanyl, (2- or 3-thienyl), quinolinyl, naphthyl, and phenyl;
R₈ is independently selected from hydrogen or R₉;
R₉ is C₁₋₄ alkyl optionally substituted by one to three fluorines;
R₁₀ is OR₈ or R₁₁;
R₁₁ is hydrogen, or C₁₋₄ alkyl unsubstituted or substituted by one to three fluorines; or when R₁₀ and R₁₁ are as NR₁₀R₁₁ they may together with the nitrogen form a 5 to 7 membered ring comprised of carbon or carbon and one or more additional heteroatoms selected from O, N, or S;
R₁₃ is a substituted or unsubstituted heteroaryl group selected from the group consisting of oxazolidinyl, oxazolyl, thiazolyl, pyrazolyl, triazolyl, tetrazolyl, imidazolyl, imidazolidinyl, thiazolidinyl, isoxazolyl, oxadiazolyl, and thiadiazolyl, and where R₁₃ is substituted on R₁₂ or R₁₃ the rings are connected through a carbon atom and each second R₁₃ ring may be unsubstituted or substituted by one or two C₁₋₂ alkyl groups unsubstituted or substituted on the methyl with 1 to 3 fluoro atoms; and
R₁₄ is hydrogen or alkyl of 1-6 carbon atoms;
which process comprises;
treating a ketone of Formula (B) 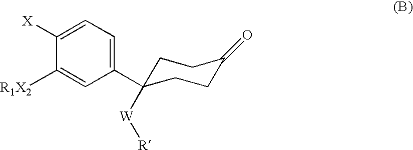
wherein X and R₁X₂ are the same as in Formula (A);
with a lower alkyldihaloacetate in a polar aprotic solvent, and
optionally saponifying the resulting alpha-haloepoxy ester.

9. The process of claim 8 wherein, in the compound of formula B, R₁ is CH₂-cyclopropyl, CH₂—C₅₋₆ cycloalkyl, or C₄₋₆ cycloalkyl and R₂ is C₁₋₂ alkyl unsubstituted or substituted by 1 or more halogens, the lower alkyldihaloacetate is lower alkyl dichloroacetate, and the base is an alkali metal t-butoxide.

10. The process of claim 9 wherein about 1.5 equivalent of the acetate and 1.5 equivalents of alkali metal t-butoxide are used.

11. The process of claim 10 wherein the acetate is methyl or ethyl dichloroacetate and the base is potassium t-butoxide.

12. The process of claim 8 wherein, in the compound of formula B, W is a bond and R′ is CN or W is —C≡C—, R₁ is cyclopentyl and R₂ is CH₃.

13. A process for enriching the cis form of a compound of Formula (IA) 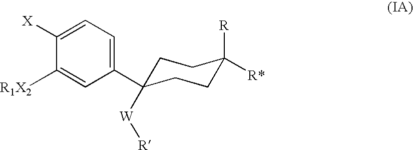
wherein:
R₁ is —(CR₄R₅)ₙC(O)O(CR₄R₅)ₘR₆, —(CR₄R₅)ₙC(O)NR₄(CR₄R₅)ₘR₆, —(CR₄R₅)ₙO(CR₄R₅)ₘR₆, or —(CR₄R₅)ᵣR₆ wherein the alkyl moieties unsubstituted or substituted with one or more halogens;
m is 0 to 2;
n is 0 to 4;
r is 0 to 6;
R₄ and R₅ are independently selected hydrogen or C₁₋₂ alkyl;
R₆ is hydrogen, methyl, hydroxyl, aryl, halo substituted aryl, aryloxyC₁₋₃ alkyl, halo substituted aryloxyC₁₋₃ alkyl, indanyl, indenyl, C₇₋₁₁ polycycloalkyl, tetrahydrofuranyl, furanyl, tetrahydropyranyl, pyranyl, tetrahydrothienyl, thienyl, tetrahydrothiopyranyl, thiopyranyl, C₃₋₆ cycloalkyl, or a C₄₋₆ cycloalkyl containing one or two unsaturated bonds, wherein the cycloalkyl or heterocyclic moiety is unsubstituted or substituted by 1 to 3 methyl groups, one ethyl group, or an hydroxyl group;
provided that:
a) when R₆ is hydroxyl, then m is 2; or
b) when R₆ is hydroxyl, then r is 2 to 6; or
c) when R₆ is 2-tetrahydropyranyl, 2-tetrahydrothiopyranyl, 2-tetrahydrofuranyl, or 2-tetrahydrothienyl, then m is 1 or 2; or
d) when R₆ is 2-tetrahydropyranyl, 2-tetrahydrothiopyranyl, 2-tetrahydrofuranyl, or 2-tetrahydrothienyl, then r is 1 to 6;
e) when n is 1 and m is 0, then R₆ is other than H in —(CR₄R₅)ₙO(CR₄R₅)ₘR₆;
X is YR₂;
Y is O;
X₂ is O;
R₂ is —CH₃ or —CH₂CH₃, optionally substituted by 1 or more halogens;
R and R* are hydrogen or C(O)E wherein one of R or R* is always hydrogen and the other is always C(O)E where E is OR₁₄ or SR₁₄ and R₁₄ is hydrogen;
W is a bond or is alkenyl of 2 to 6 carbon atoms or alkynyl of 2 to 6 carbon atoms;
when W is a bond R′ is hydrogen, halogen, C₁₋₄ alkyl, CH₂NHC(O)C(O)NH₂, halo-substituted C₁₋₄ alkyl, CN, OR₈, CH₂OR₈, NR₈R₁₀, CH₂NR₈R₁₀, C(Z′)H, C(O)OR₈, or C(O)NR₈R₁₀; and
when W is alkenyl of 2 to 6 carbon atoms or alkynyl of 2 to 6 carbon atoms then R′ is R′ is COOR₁₄, C(O)NR₄R₁₄ or R₇;
R₇ is —(CR₄R₅)_{q}R₁₂ or C₁₋₆ alkyl wherein the R₁₂ or C₁₋₆ alkyl group is unsubstituted or substituted one or more times by: methyl or ethyl unsubstituted or substituted by 1-3 fluorines, —F, —Br, —Cl, —NO₂, —NR₁₀R₁₁, —C(O)R₈, —CO₂R₈, —O(CH₂)₂₋₄OR₈, —O(CH₂)_{q}R₈, —CN, —C(O)NR₁₀R₁₁, —O(CH₂)_{q}C(O)NR₁₀R₁₁, —O(CH₂)_{q}C(O)R₉, —NR₁₀C(O)NR₁₀R₁₁, —NR₁₀C(O)R₁₁, —NR₁₀C(O)OR₉, —NR₁₀C(O)R₁₃, —C(NR₁₀)NR₁₀R₁₁, —C(NCN)NR₁₀R₁₁, —C(NCN)SR₉, —NR₁₀C(NCN)SR₉, —NR₁₀C(NCN)NR₁₀R₁₁, —NR₁₀S(O)₂R₉, —S(O)_{m′}R₉, —NR₁₀C(O)C(O)NR₁₀R₁₁, —NR₁₀C(O)C(O)R₁₀, or R₁₃;
q is 0, 1, or 2;
R₁₂ is R₁₃, C₃-C₇ cycloalkyl, or an unsubstituted or substituted aryl or heteroaryl group selected from the group consisting of (2-, 3- or 4-pyridyl), pyrimidyl, pyrazolyl, (1- or 2-imidazolyl), pyrrolyl, piperazinyl, piperidinyl, morpholinyl, furanyl, (2- or 3-thienyl), quinolinyl, naphthyl, and phenyl;
R₈ is independently selected from hydrogen or R₉;
R₉ is C₁₋₄ alkyl optionally substituted by one to three fluorines;
R₁₀ is OR₈ or R₁₁;
R₁₁ is hydrogen, or C₁₋₄ alkyl unsubstituted or substituted by one to three fluorines; or when R₁₀ and R₁₁ are as NR₁₀R₁₁ they may together with the nitrogen form a 5 to 7 membered ring comprised of carbon or carbon and one or more additional heteroatoms selected from O, N, or S;
R₁₃ is a substituted or unsubstituted heteroaryl group selected from the group consisting of oxazolidinyl, oxazolyl, thiazolyl, pyrazolyl, triazolyl, tetrazolyl, imidazolyl, imidazolidinyl, thiazolidinyl, isoxazolyl, oxadiazolyl, and thiadiazolyl, and where R₁₃ is substituted on R₁₂ or R₁₃ the rings are connected through a carbon atom and each second R₁₃ ring may be unsubstituted or substituted by one or two C₁₋₂ alkyl groups unsubstituted or substituted on the methyl with 1 to 3 fluoro atoms;
which process comprises treating the lower alkyl ester, lower alkyl thioester or mixed anhydride of Formula (IA) with an alkoxide base.

14. The process of claim 13 wherein the compound of formula IA, R₁ is CH₂-cyclopropyl, CH₂—C₅₋₆ cycloalkyl, or C₄₋₆ cycloalkyl, R₂ is C₁₋₂ alkyl unsubstituted or substituted by 1 or more halogens, the base is a alkali metel t-butoxide, and the reaction runs for 5-24 hours.

15. The process of claim 13 wherein the compound of formula IA is [4-cyano-4-(3-cyclopentyloxy-4-methoxyphenyl)cyclohexane-1-carboxylic acid].

16. The process of claim 13 wherein the base is potassium t-butoxide.

17. A compound which is lower alkyl 2-chloro-6-cyano-6-[3-(cyclopentyloxy)-4-methoxyphenyl]-1-oxaspiro[2.5]octane-2-carboxylate.

18. The compound of claim 17 which is methyl 2-chloro-6-cyano-6-[3-(cyclopentyloxy)-4-methoxyphenyl]-1-oxaspiro[2.5]octane-2-carboxylate.

19. A compound which is 2-chloro-6-cyano-6-[3-(cyclopentyloxy)-4-methoxyphenyl]-1-oxaspiro[2.5]octane-2-carboxylic acid.
